(19) United States
(12) Patent Application Publication
(10) Pub. No.: US 2019/0008905 A1
(43) Pub. Date: Jan. 10, 2019

(54) COMPOSITIONS FOR MANAGEMENT OF WOUNDS, SKIN DISEASES, DEHYDRATION, CHRONIC DISEASES, AND RESPIRATORY DISEASES

(71) Applicant: Noori AL-WAILI, Richmond Hill, NY (US)

(72) Inventor: Noori AL-WAILI, Richmond Hill, NY (US)

(21) Appl. No.: 15/864,311

(22) Filed: Jan. 8, 2018

Related U.S. Application Data
(60) Provisional application No. 62/529,615, filed on Jul. 7, 2017.

(51) Int. Cl. A61K 35/644; A61K 45/06; A61K 36/71; A61K 36/48; A61K 36/88; A61K 36/63; A61K 31/12; A61K 36/55; A61K 36/82; A61K 36/906; A61K 31/122; A61P 17/02; A61P 17/00; A61P 17/06; A61P 37/08; A61P 31/22; A61P 33/02; A61P 31/04; A61P 1/04; A61P 3/10; A61P 13/02; A61P 7/06; A61P 3/06; A61P 9/00; A61P 13/12; A61P 9/12; A61P 37/00; A61P 3/00; A61P 11/00

(52) U.S. Cl. CPC A61K 35/644; A61P 11/00; A61K 36/71; A61K 36/48; A61K 36/88; A61K 36/63; A61K 31/12; A61K 36/55; A61K 36/82; A61K 36/906; A61K 31/122; A61P 17/02; A61P 17/00; A61P 17/06; A61P 37/08; A61P 31/22; A61P 33/02; A61P 31/04; A61P 1/04; A61P 3/10; A61P 13/02; A61P 7/06; A61P 3/06; A61P 9/00; A61P 13/12; A61P 9/12; A61P 37/00; A61P 3/00; A61K 45/06

(57) ABSTRACT

A composition including a mixture of at least two different honeys is used to treat a variety of medical conditions. The composition can be administered topically, orally, by inhalation or parenterally. The composition is useful in treating wounds, infections, diabetes, skin conditions, renal conditions, dehydration, malnutrition, hypertension and various other conditions. It has been found that the mixture of at least two different types of honey, either alone or in combination with other bee products is more effective than the administration of single honey.

### CROSS-REFERENCE TO RELATED APPLICATIONS

This application claims priority under 35 USC 119(e) of U.S. Provisional Application No. 62/529,615, filed on Jul. 7, 2017, the disclosure of which is herein incorporated by reference.

### BACKGROUND OF THE INVENTION

### 1. Field of the Invention

The present invention relates to compositions for management of wounds, skin diseases, dehydration, chronic diseases, and respiratory diseases. In particular, the invention relates to using mixed honey, either alone or in combination with other products for the treatment of various afflictions of the human and animal body.

### 2. The Prior Art

It is well known that various bee products have healing properties and are suitable for treating wounds, skin diseases and other afflictions.

Honey has served as a common base for wound healing compositions since ancient times. Honey has been found to have antibacterial, antifungal, antiviral, anti-inflammatory, antihypertensive, antioxidant, antitumor, cardioprotective, hepatoprotective, and hypoglycemic properties. It has been found that there is a large amount of variation in potency of antibacterial and antioxidant activity between different honeys, most likely due to varying levels of bioactive factors in honey.

Propolis is a natural plant product collected by Apis mellifera honeybees from plants. It is a resinous mixture of botanical balsams and resin with digestive enzymes of bees. It contains more than 300 natural compounds including polyphenols, phenolic aldehydes, amino acids, steroids, sequiterpene-quinones, coumarins, and inorganic compounds. The chemical composition and biological properties of propolis depend on phytogeographical areas, seasonal collection time, and botanical sources. Propolis has no constant chemical composition. Depending on its region of origin, this substance consists of approximately 55% resinous and balm substances, approximately 30% wax, 5 to 10% essential oils, 2 to 5% pollen, vitamins and microelements. Therefore, mixing of propolis certainly will result in.superpropolis. Propolis is relatively non-toxic, with a no-effect level in a dose of 1400 mg/kg body weight/day (Burdock G., Review of the biological properties and toxicity of bee propolis (propolis). Food Chem Toxicol 1998;36: 347-363).

Propolis has antioxidant, immune-modulatory effects, antimicrobial, anti-inflammatory, antitumor, antiulcer, hepato and cardio-protective properties. Propolis also has antimicrobial activity and has renal and hepatic protection property.

CAPE (caffeic acid phenethyl ester) is an active phenolic part of propolis and has antitumoral, antiproliferative, anti-inflammatory, antineoplastic, and antioxidant properties. The molecular weight and empirical formula of CAPE are 284.3 g/mol and C₁₇H₁₆O₄. All flavonoids within propolis, but not CAPE, are reported to have a low order of acute oral toxicity with a reported LD50 of 8-40 g/kg. Similarly, a safe dose in humans is estimated as 1.4 mg/kg body weight/day, or approximately 70 mg/day (Burdock G., Review of the biological properties and toxicity of bee propolis (propolis). Food Chem Toxicol 1998; 36: 347-363).

Garlic (Allium sativum L) is one of the world's oldest medicines and it has biological activities, including anti-carcinogenic, antiatherosclerotic, antithrombotic, antimicrobial, antiinflammatory and antioxidant effects.

Curcumin belongs to the family of natural compounds collectively called curcuminoids and it possesses remarkable beneficial anti-oxidant, anti-inflammatory, anti-cancer, and neuroprotective properties. It is the most active component of rhizome of Curcuma longa L. (common name: turmeric). Furthermore, curcumin is effective in the treatment of chronic pain, inflammatory dermatoses, acceleration of wound closure, skin infections, as well as dyspigmentation.

Olive oil contains 95 to 99% acyl glyceroles, 0.5 to 1.5% uns.aponifyable substances and 0.1 to 3% free fatty acids, glycerides, secciridoids and flavonoids. The main fatty acids are oleic acid, palmitic acid and linoleic acid. Furthermore, it contains phenolic compounds, carbohydrates, steroles, triterpene alcohols, hydroxy triterpenic acids, tocopheroles, phospholipids, carotinoids, chlorophyll and pheophytines.

Nigella sativa belongs to the botanical family of Ranunculaceae and commonly grows in the Eastern Europe, Middle East, and Western Asia. It is a small shrub with tapering green leaves and rosaceous white and purplish flowers. Its ripe fruit contains tiny seeds, dark black in color. Nigella sativa is native to Southern Europe, North Africa and Southwest Asia, Middle Eastern Mediterranean region, India, Pakistan, Syria, and Turkey. Among Muslims, it is considered as one of the greatest forms of healing medicine available as was mentioned that black seed is the remedy for all diseases except death in one of the Prophetic hadith. It is also recommended for use on regular basis in Tibb-e-Nabwi (Prophetic Medicine). Nigella sativa seeds contain an essential oil (0.4-2.5%), fixed oil (36-48%), alkaloids, saponin and proteins. Nigella sativa oil was shown to contain thymoquinone which is the main bioactive component (27.8-57.0%) of the essential oil of the black seed. Toxicity studies on laboratory animals have reported that nigella sativa oil and thymoquinone are quite safe, mainly when given orally.

Data has showed that nigella sativa has diuretic, antihypertensive, antidiabetic, anticancer and immunomodulatory, analgesic, antimicrobial, anthelmintics, analgesics and anti-inflammatory, spasmolytic, bronchodilator, gastroprotective, hepatoprotective, renal protective and antioxidant properties. The seeds of nigella sativa are widely used in the treatment of various diseases like bronchitis, asthma, diarrhea, rheumatism and skin disorders. It is also used as liver tonic, digestive, anti-diarrheal, appetite stimulant, to increase milk production in nursing mothers to fight parasitic infections, and to support immune system. Nigella sativa oil and thymoquinone have been used as anti-inflammatory, antioxidant and anticancer therapeutic Agents.

Bee venom therapy is thousands of years old and it involves the application of live bee stings to the skin or the injection of bee venom into the skin. Bee venom is produced by honeybees (Apis mellifera). Bee venom has anti-inflammatory, antibacterial, and anti-rheumatic activities. It has been used in arthritis, rheumatism, back pain, multiple sclerosis, cancer, and skin diseases. It relieves pain and inflammation and it has an immune response enhancing effect. It contains melittin, apamin, adolapin, phospholipase A2, mast cell degranulating peptide, α-D-glucosidase, biologically activity amines, hyaluronidase, acid phosphomonesterase, and lysophospholipase. Melittin has anti-inflammatory and antioxidant activity.

Syzygium aromaticum (synonym: Eugenia cariophylata) is commonly known as clove. It is a medium sized tree (8-12 m) native from the Maluku islands in east Indonesia. Cloves are the aromatic flower buds of a tree in the family Myrtaceae. Clove represents one of the major vegetal sources of phenolic compounds as flavonoids, hidroxibenzoic acids, hidroxicinamic acids and hidroxiphenyl propens. Eugenol is the main bioactive compound of clove. Other phenolic acids found. in clove are the caffeic, ferulic, elagic and salicylic acids. Flavonoids as kaempferol, quercetin and its derivates (glycosilated) are also found in clove in lower concentrations. Concentrations up to 18% of essential oil can be found in the clove flower buds. It was estimated that 89% of the clove essential oil is eugenol and 5% to 15% is eugenol acetate and β-cariofileno.

The essential oil extracted from flower buds of clove is used as a topical application to relieve pain and to promote healing. Its main constituents are phenylpropanoids such as carvacrol, thymol, eugenol and cinnamaldehyde. In addition to its antimicrobial, antioxidant, antifungal and antiviral activity (herpes simplex), clove essential oil possesses anti-inflammatory, cytotoxic, insect repellent and anesthetic properties. Syzygium aromaticum (clove) has an anti-microbial activity.

Acacia Arabica gum is a traditional oral hygiene substance that has been used for centuries by many communities in the Middle East and North Africa. It consists mainly of Arabica, a complex mixture of the calcium, magnesium and potassium salts of Arabic acid. There are also other constituents such as tannins, cyanogenic glycosides, oxidases, peroxidases and pectinases; all of which have been shown individually to exhibit antimicrobial properties.

Gum Arabic is mixture of polysaccharides, oligosaccharides and glycoproteins. It is exudates of Acacia Senegal/seyal trees. It is water soluble; therefore, it is used as an emulsifier, thickening substance and flavor stabilizer in many pharmaceutical and food industries.

Bees collect pollen from plant anthers, mix it with a small dose of the secretion from salivary glands or nectar, and place it in corbiculae which are situated on the tibia of their hind legs. The chemical composition of bee pollen depends strongly on the plant source and geographic origin, together with other factors such as climatic conditions, soil type, and bees' race and activities. Bee pollen contains about 250 substances including amino acids, lipids (triglycerides, phospholipids), protein, vitamins, macro- and micronutrients, phenolic compounds, enzymes, and coenzymes and flavonoids.

Bee pollen contains 22.7% of protein on average (10.4% of essential amino acids, significant amounts of nucleic acids), digestible carbohydrates (30.8% on average), essential fatty acids, phenolic compounds (flavonoids, leukotrienes, catechins, and phenolic acids), multivitamins 0.7%, calcium, phosphorus, magnesium, sodium, and potassium, iron, copper, zinc, manganese, silicon, and selenium 1.6%. Bee pollen has antifungal, antimicrobial, antiviral, anti-inflammatory, immune-stimulating, and local analgesic and also facilitates the granulation process of the burn wound healing.

Royal jelly is a mixture of yellow white creamy and acidic secretions produced by the worker honeybees using their mandibular and hypopharingeal glands to supply food for a queen honeybee. It is rich in protein, amino acids, fatty acids, calcium, iron, minerals, vitamins, and carbohydrates. 10-HAD is the most active ingredient. Royal jelly has antimicrobial properties against yeast, Gram-negative and Gram-positive bacteria. Royal jelly stimulates immune-co-potent cell proliferation and production of antibodies in mice.

Flaxseed (linseed, Linum usitatissimum), is the richest plant source of a-linolenic acid (50%-62% of flaxseed oil, or 22% of whole flaxseed) and lignans (a class of phytoestrogen, range: 0.2-13.3 mg/g flaxseed) and it contains dietary fiber (28% by weight), a third of which is soluble fiber. Whole flaxseed or flaxseed oil are included in the general oral composition and renal healing composition.

Juglans regia Linn. (Juglandaceae) is a valuable medicinal plant and various parts of the plant have been used for management of diseases in different cultures. Walnuts contain unsaturated fatty acids, proteins, fiber, vitamins, minerals, phytosterols and polyphenols. Furthermore, walnuts contain a number of potentially neuroprotective compounds like vitamin E, folate, melatonin, several antioxidative polyphenols and significant amounts of ω-3 fatty acids. Its health benefits include reduced risk of cardiovascular disease, certain cancer and type II diabetes, and amelioration of symptoms of neurological disorders. It is rich in polyphenols and other phytochemicals, polyunsaturated and fatty acids with a particularly high ω3: ω6 ratio-the highest among all the tree nuts.

While each of these natural ingredients alone have healing properties, it would be desirable to produce a product that increases the effectiveness of the natural ingredients beyond what has already been documented.

### SUMMARY OF THE INVENTION

It is an object of the invention to provide a healing substance that exhibits superior healing properties as compared to the natural products alone described above.

This object is accomplished by a product that contains a mixture of different types of the same substance, namely honey. By mixing different honeys, the various beneficial properties of the different honeys are combined and enhance the healing process.

The different honeys can be honeys of different color such as mixing dark honey with light honey, yellow honey or white honey, or with any other color; of different floral origin either unifloral or multiflora, and can be the same honey made by different bee species. Also, the honeys can be of same plant or floral origin but made from different bee species, or can be different honeys, unifloral or multiflora, but all made by same species or different species. The invention can include mixing unifloral honey with unifloral honey, or mixing unifloral honey with multiflora honey. The honeys can be from same geographical location, or from different geographical locations or countries or from different seasons. The honeys can be mixed in any desired proportions, such as 1:1 ratio (generally 45-55 wt % each). Other ratios could also be used. The formulations can include total honey or honey extract, such as water, alcohol or ethyl acetate extracts.

The mixed honey formulations are useful for the prevention/treatment of, inter alia, wounds, ulcers, burns, skin infection, and related skin disorders such as contact dermatitis, eczema, psoriasis, wrinkle and other skin diseases (inflammatory, infectious or allergic). The compositions are also antimicrobial to fungus, mold, bacteria, viruses and yeasts, indicating potential to treat skin, wound and ulcer infections in human or animals. The invention also comprises oral compositions for treatment of, inter alia, anemia, enhancement of immunity, potentiating the effects of antibiotics and chemotherapy, for management of dyslipidemia, diabetes mellitus, cardiovascular diseases, H. pylori gastritis or ulcers, dyspepsia, acute and chronic kidney diseases and hypertension, and for prevention or amelioration of toxic effects of heavy metals or chemotherapies or radio therapy.

The invention also comprises intravenous compositions for protection of, inter alia, liver, kidney, bone marrow when used with chemotherapy, cytotoxic drugs, and immunosuppressive agents, or used in inflammatory diseases, infectious diseases or sepsis or dehydration or malnutrition or part of cancer management.

The invention also comprises inhalation compositions for the management of infectious respiratory diseases, sinusitis, and lung obstructive diseases or malignancies.

Although honey, propolis, royal jelly and bee pollen alone or mixed with other substances are already known in the medical science, yet so far mixtures of different honeys has never been used in topical, parenteral, inhalational or oral treatment.

Honey has no constant chemical composition, which is dependent on the region of origin, seasons, and plant origin. The functional properties therefore are different in their potency. The different compositions and properties can be utilized further by mixing samples of honeys collected from different regions or plant source to provide new honey with higher nutritional values and more potent medicinal applications.

The objective of the present invention is to provide a natural agent for nutritional and medicinal use, which has a broad application in human and animals, which also composed of multiple natural ingredients and, therefore, is lack of adverse effects, without contraindication, and well tolerated.

A further objective of the present invention is to use the natural ingredients in doses, which are suitable for human or animal use and without side effect.

A particular advantageous use of the mixed honey, alone or in combination with one or more than one of the following natural substances such as propolis, bee pollen, royal jelly, nigella sativa, thymoquinone, CAPE, curcumin, bee venom or melittin, garlic or aged garlic or aged garlic extract (AGE), flaxseed or flaxseed oil, curcumin, green tea, fenugreek seeds, walnuts, ginger, acacia Arabica, clove or olive oil will provide a composition with high potency, wide spectrum of medicinal and nutritional u\grave{s}e, low cost, lack of adverse effects, well tolerability, ability to potentiate the use of chemotherapy, antibiotics and other modern interventions, improving circulation, and ameliorating side effects of immunosuppressive and chemotherapeutic agents as well as radiotherapy.

A further advantageous use of the substance mixture according to the invention is that treatment with these substances includes a decrease in length of hospital stay, decrease in complications of chronic diseases such as diabetic nephropathy, diabetic retinopathy, hypertensive heart disease, burn scars and deformities, wound scars, and decreasing further invasive interventions.

A further advantageous use of the substance mixture according to the invention consists in preparing compositions for, not limited to, skin care, skin diseases, diabetes mellitus, hypertension, .cardiovascular diseases, kidney diseases, urinary calculus, infections (bacterial, viral and parasitic), malignancies, malnutrition, and respiratory diseases.

A further advantageous use of the substance mixture according to the invention consists in preparing compositions for to be used in topical, parenteral, inhalational or oral treatment. Another advantageous use of the substance mixture according to the invention includes providing biological systems with potent antioxidant capacity, potent antimicrobial intervention, potent anti-inflammatory agents, potent analgesic intervention, and potent nutritional support more than using individual ingredients. There is no intervention in modern medicine available with all these properties together.

Further, it is an object of the present invention to provide a composition with a base of mixed honey, which is suitable for the treatment of parasitic diseases such as Entamoeba histolytica, Giardia lamella, Blastocystis species, schistosomiasis, malaria, flukes, trypanosome cruzi, hydatid cyst, toxoplasmosis, visceral leishmaniasis, ascaris, nematodes, trematodes, and trichomonas species. The formulations can include total honey or honey extract, such as water, alcohol or ethyl acetate extracts.

The different compositions can be administered topically, orally, parenterally, by inhalation (nasal or oral), vaginally, or rectally in dosage unit formulations containing conventional nontoxic pharmaceutically acceptable carriers, adjuvants, and vehicles, as desired. The term parenteral as used herein includes intravenous, intramuscular, intraperitoneal, intrasternal injection, or infusion techniques.

The wound/skin healer composition is a composition for topical application in or on a wound or ulcer that is believed to promote healing and tissue growth, provide nutritional factors and antioxidants, and to eradicate infection and accelerate wound healing. The composition includes mixed honey, either alone or combined with, propolis extract or powder, CAPE, mixed propolis extract or powder, bee venom or melittin, bee pollen or mixed bee pollen, royal jelly or mixed royal jelly, curcumin, acacia Arabic, olive oil, Nigella Sativa or thymoquinone, clove, and garlic or aged garlic or AGE. Acacia Arabica is used in heavy exudate, bleeding wounds, and also to increase viscosity of the formulae. It has antioxidant and antimicrobial effects. Multiple compositions can be prepared with mixing any two or more ingredients including honey or not including honey to achieve the same goals.

The powders of crude ingredients or their extracts are mixed into the viscous honey base, and the composition may be applied directly to the wound or ulcer or skin diseases, or the composition may be applied to sterile gauze or incorporated in any wound materials such as alginate, foams, hydrocolloids, interactive dressing, or composite dressing and the wound or ulcer, particularly an open surgical wound or deep ulcer, may be packed with the gauze.

The amount of the wound/skin compositions to be administered and the frequency of administration and period of the treatment depends on the type, location, chronicity, and severity of the wound, ulcer or infection. The compositions may be presented in unit-dose or multi-dose containers. And they may include additional ingredients that are conventional in the art with regard to topical formulations. The healing herein includes curing, alleviating, reversing, palliative and prophylactic treatment of the condition.

The main advantage of the mixed honey composition according to the invention includes:

1—Natural products and available everywhere,

2—Safe and almost no side effects,

3—Synergistic effects as a wound healer as well as antimicrobial to eradicate infections,

4—Provides wound and skin with excellent nutritional, anti-inflammatory and antioxidants ingredients, and

5—Debridement of dead tissue in wounds and ulcers.

The products are for wound and ulcer treatment, which consist of multi component combination of natural substances. This .composition allows batch process production, where it is possible to accomplish mixing and fusion of various natural substances, and cream homogenization, by converting all substances in the dissolvable stage. Honey is used as a semiliquid and can be combined with other ingredients. Hard, propolis is converted into dissolvable stage by extraction with ethanol, obtain water extract, or could be used as ground powder. Bee pollen powder is dried and ground into powder. Other ingredients are used as ground powder, or water and alcohol extracts or as an oil form.

One or more pharmaceutically acceptable diluents, excipients or carriers can be added. The composition may comprise substances which assist in its application or storage stability, such as, pharmaceutical adjuvants, stabilizers, buffer substances, preservatives, water, thickening agents, emulsifiers, wax, beeswax, and the like.

Gels, ointments and creams may be formulated, for example, with an aqueous or oily base with the addition of suitable thickening. Lotions may be formulated with an oily or aqueous base and can contain one or more emulsifying agents, dispersing agents, stabilizing agents, coloring agents, and/or suspending agents and thickening agents.

Suitable carriers also include creams/ointments conventionally used for topical pharmaceutical preparations, such as alginate, preservatives such as benzyl alcohol, buffers to control pH such as disodium hydrogen phosphate/sodium dihydrogen phosphate, agents to adjust osmolarity such as sodium chloride, and stabilizers such as EDTA.

The composition can be applied directly to the wound as a gel, ointment, liquid, cream, or the like as described above. Alternatively, the composition is administered in the form of a wound dressing. Wound dressing may include any one of the different types of substrates and/or backings that are commercially available, including films such as polyurethane films, hydrocolloids such as hydrophilic colloidal particles bound to polyurethane foam, hydrogels, foams such as hydrophilic or hydrophobic, calcium alginates, and cellophane. For example, the composition can be applied to the surface of, or incorporated into a dressing gauze or matrix. Suitable gauze dressings may include, for example, a swabs, dry woven or non-woven sponges, bandages and wraps with varying degrees of absorbency. Exemplary fabric compositions may include cotton, rayon or polyester. In certain embodiments, gauzes and non-woven dressings may be available sterile and with or without an adhesive border. In certain embodiments, the dressings also comprise one or more additional pharmaceutically active compounds and/or carrier agents, such as xeroform, oil, saline, zinc salts, petrolatum, and scarlet red. The agent to be used according to the invention may be prepared in all the application forms common for topical medicaments, such as an ointment, shampoo emulsion, stocking, sponge, soap, facial pack, lotion, bath salts, or plaster.

The present composition and/or the components of the composition may be sterilized by any suitable method, including conventional, well-known sterilization techniques, such as autoclaving. Raw, unheated honey can be sterilized, for example, by ozonizing the honey.

The dosage regimen for treating wounds is selected in accordance with a variety of factors including the age, weight, sex, and medical condition of the patient, the severity of the wound, the route of administration, pharmacological considerations such as the activity, efficacy, pharmacokinetic and toxicology profiles of the particular composition used, whether a dressing or drug delivery system is used and whether the composition is administered as part of a drug combination.

The advantageous use of the substance mixture according to the invention consists in preparing an agent for wound and ulcer healing. These properties include anti-inflammatory, antibacterial, and antimycotic effects; wound and ulcer healing activity; supplying vitamins, trace elements, natural glucose, and amino acids. The mixture will provide stronger antioxidants, anti-inflammatory and antimicrobial activities as compared to single ingredient treatments.

The oily content of the compound acts as a barrier, preventing excessive loss of body fluids and decreasing evaporative wound water loss thus preventing wound desiccation and providing optimal wet atmosphere for wound healing. Olive oil contains polyphenolic compounds, which has an antibacterial effect to enhance wound healing. The compositions may include deodorized garlic (Allium sativum), such as that described in (U.S. Pat. No. 4,933,201 to Sakai) or pure, powered, pulverized, minced or extract, preferably obtained from the bulb, but not limited to any portion of the plant, preferably derived from the aforementioned member of the genus, but not limited to any species within the Allium genus such as Allium oleraceum, Allium ursinum, Allium ampeloprasum and Allium canadense. The compositions may include pure, solids, decolorized or extract of Turmeric Rhizome (Curcuma Longa), preferably derived from this member of the genus, but not limited to variation in species of the curcuma genus such as Curcuma aromatica and Curcuma amada.

The general oral healing composition includes mixed honey alone or combined with bee pollen or mixed bee pollen, royal jelly or mixed royal jelly, flaxseed, acacia Arabica, curcumin, Nigella sativa or thymoquinone or Nigella sativa oil, garlic or aged garlic or AGE, olive oil, cloves or clove extract or clove oil, propolis or mixed propolis or their extracts or CAPE, Flaxseed or flaxseed oil, green tea or green tea extract, walnuts of walnuts oil, ginger or ginger extract, fenugreek seeds or extract, date seeds, why protein, peanuts, for fish oil for treatment of, not limited to, anemia, enhancement of immunity, potentiating the effect of antibiotics and chemotherapy, for management of dyslipidemia, diabetes mellitus, hypertension, and for prevention or amelioration of toxic effects of heavy metals or chemotherapies. Multiple compositions can be prepared with mixing any two or more ingredients including honey or not including honey to achieve the same goals. The formulations can include total honey or honey extract, such as water, alcohol or ethyl acetate extracts.

The oral healing composition is a product for oral use to maintain or potentiate the anti-oxidant levels in healthy individuals or in patient during treatment with a chemotherapeutic agent, antibiotics or pain medications such as NSAIDs.

Other aspects of the invention also relate to methods to reduce the toxicity of exposure of antibiotics or cytotoxic agents in a patient, and heavy metals in a patient or healthy individual comprising administering the healing oral formulae to the patient or normal individual with such exposures.

An important aspect relates to a method of reducing the dose of antibiotic or cytotoxic agents administered to a patient for purpose of management of infection, sepsis or malignancies, due to synergistic effects, anti-inflammatory, antioxidant properties and enhancement of immunity, comprising administering the healing oral compositions to the patient.

The compositions may be presented in unit-dose or multi-dose containers. And they may include additional ingredients that are conventional in the art with regard to oral formulations. The healing herein includes curing, alleviating, reversing, palliative and prophylactic treatment of the condition.

The compositions can be administered orally in dosage unit formulations containing conventional nontoxic pharmaceutically acceptable carriers, adjuvants, and vehicles, as desired. One or more pharmaceutically acceptable diluents, excipients or carriers can be added. The composition may comprise substances which assist in its application or storage stability, such as, pharmaceutical adjuvant, stabilizers, buffer substances, preservatives, water, thickening agents, emulsifiers, beeswax, and the like. The advantageous use of the mixture according to the invention consists in preparing an agent for increasing immunity, antioxidant capacity and healing cellular power, and also provides an excellent nutritional support in normal individuals and in patients with chronic diseases and infections. The mixture will provide stronger antioxidants, anti-inflammatory and antimicrobial than individual ingredient. These ingredients have anti-cancer properties and immune system stimulating activity.

The main advantages include:

1—Natural products and available everywhere,

2—Safe and almost no side effects,

3—Synergistic effects as a disease healer as well as an antimicrobial to eradicate infections, and

4—Provides all body system with excellent nutritional, anti-inflammatory and antioxidants ingredients.

The invention also relates to a renal healing composition, wherein the mixed honey is mixed with propolis or mixed propolis or propolis or mixed propolis extracts or CAPE, flaxseed, acacia Arabica, curcumin or curcumin extract, royal jelly or mixed royal jelly, olive oil, ginger or ginger extract, green tea or green tea extract, bee pollen or mixed bee pollen, garlic or aged garlic or AGE or garlic oil, Nigella sativa or nigella sativa oil or thymoquinone for management of, not limited to, kidney diseases, proteinuria and urinary calculus, and is used as an oral recipe. The dose depends on the stage of the kidney disease, severity of proteinuria, etiology of proteinuria and the comorbidities. The composition presumably alleviates or cures proteinuria, and prevents kidney stone formation and facilitates urinary calculus passage. This composition provides a diuretic effect, antioxidants, anti-inflammatory action and analgesic effect. Multiple compositions can be prepared with mixing any two or more ingredients including honey to achieve the same goals. The formulations can include total honey or honey extract, such as water, alcohol or ethyl acetate extracts.

The compositions may be presented in unit-dose or multi-dose containers. And they may include additional ingredients that are conventional in the art with regard to oral formulations. The healing herein includes curing, alleviating, reversing, palliative and prophylactic treatment of the condition. The comₚositions can be administered orally in dosage unit formulations containing conventional nontoxic pharmaceutically acceptable carriers, adjuvants, and vehicles, as desired. One or more pharmaceutically acceptable diluents, excipients or carriers can be added. The composition may comprise substances which assist in its application or storage stability, such as, pharmaceutical adjuvants, stabilizers, buffer substances, preservatives, water, thickening agents, emulsifiers, beeswax, and the like. The advantageous use of the substance mixture according to the invention consists in preparing the product or composition that will be useful for management of, inter alia, proteinuria due to diabetes, nephritis, nephrotic syndrome, or due to any reason such as heavy metal intoxication, vasculitis, systemic lupus, IgA nephropathy, membranous nephropathy and others causes mentioned in the medical literature.

The mixture will provide stronger antioxidants, anti-inflammatory and antimicrobial than individual ingredients. The treatment goals of this mixture are:

1—healing of proteinuria,

2—healing of kidney injury in acute and chronic kidney diseases, and

3—as part of management of kidney and urinary calculus and crystaluria.

The main advantages include:

1—Natural products and available everywhere,

2—Safe and almost no side effects,

3—Synergistic effects as kidney healer as well as antimicrobial to eradicate infections, and

4—Provides urinary system with excellent nutritional, anti-inflammatory and antioxidants ingredients.

The invention also relates to a H. pylori healing composition comprising mixed honey alone or in combination with propolis or mixed propolis or CAPE, acacia Arabica, garlic, Nigella sativa or Nigella sativa oil or thymoquinone, aged garlic or AGE or garlic oil, bee pollen or mixed bee pollen, olive oil, curcumin or curcumin extract, fish oil and cloves or cloves extract in form of oral recipe for management of H.pylori infection. The formulae could be used alone or in combination with antibiotics and a pump inhibitor. The healing herein includes curing, alleviating, reversing, palliative and prophylactic treatment of the condition. The compositions may be presented in unit-dose or multi-dose containers. And they may include additional ingredients that are conventional in the art with oral formulations. The formulations can include total honey or honey extract, such as water, alcohol or ethyl acetate extracts.

The compositions can be administered orally in dosage unit formulations containing conventional nontoxic pharmaceutically acceptable carriers, adjuvants, and vehicles, as desired. One or more pharmaceutically acceptable diluents, excipients or carriers can be added. The composition may comprise, substances which assist in its application or storage stability, such as, pharmaceutical adjuvant, stabilizers, buffer substances, preservatives, water, thickening agents, emulsifiers, beeswax, and the like. The advantageous use of the substance mixture according to the invention consists in preparing the product or composition that will be useful to eradicate H. pylori infection, peptic ulcer and dyspepsia. Multiple compositions can be prepared with mixing any two or more ingredients including honey or not including honey to achieve the same goals.

The main advantages include:

1—Natural products and available everywhere;

2—Safe and almost no side effects;

3—Synergistic effects to heal H. pylori infection; and

4—Provides the gastrointestinal tract with excellent nutritional, anti-inflammatory and antioxidants ingredients.

The invention also relates to a probiotic enhancing composition, wherein the use of mixed honey alone or mixed with nigella sativa or thymoquinone or nigella sativa oil, acacia Arabica, royal jelly or mixed royal jelly, garlic or aged garlic or garlic oil, propolis or mixed propolis, bee pollen or mixed bee pollen, olive oil to stimulate growth of gut bacteria particularly in patients using antibiotic or in patients with colitis. The compositions may be presented in unit-dose or multi-dose containers. And they may include additional ingredients that are conventional in the art with regard to oral formulations. The healing herein includes curing, alleviating, reversing, palliative and prophylactic treatment of the condition. Multiple compositions can be prepared with mixing any two or more than two of the ingredients not including honey to achieve the same goals. The compositions can be administered orally in dosage unit formulations containing conventional nontoxic pharmaceutically acceptable carriers, adjuvants, and vehicles, as desired. One or more pharmaceutically acceptable diluents, excipients or carriers can be added. The composition may comprise substances which assist in its application or storage stability, such as, pharmaceutical adjuvant, stabilizers, buffer substances, preservatives, water, thickening agents, emulsifiers, beeswax, and the like. The advantageous use.of the substance mixture according to the invention consists in preparing the product or composition that will be useful for stimulation of beneficial gut microorganisms in normal individuals and in patients with chronic diseases, on antibiotic management or having colitis or gastrointestinal infections. The formulations can include total honey or honey extract, such as water, alcohol or ethyl acetate, extracts.

The main advantages include:

1—Natural products and available everywhere;

2—Safe and almost no side effects;

3—Synergistic effect to stimulate beneficial gut microbial growth; and

4—Provides gastrointestinal tract with excellent nutritional, anti-inflammatory and antioxidant ingredients.

The invention also relates to an inhalation healing composition, wherein another aspect of the invention relates to use of the mixed honey with or without propolis or mixed propolis in an inhalation composition for management of respiratory diseases. The compositions may be presented in unit-dose or multi-dose containers. And they may include additional ingredients that are conventional in the art with inhalation formulations. The compositions can be administered by inhalation in dosage unit formulations containing conventional nontoxic pharmaceutically acceptable carriers, adjuvants, and vehicles, as desired. One or more pharmaceutically acceptable diluents, excipients or carriers can be added. The composition may comprise substances which assist in its application or storage stability, such as, pharmaceutical adjuvant, stabilizers, buffer substances, preservatives, water, thickening agents, emulsifiers, beeswax, and the like. The advantageous use of the substance mixture according to the invention consists in preparing the product or composition that will be useful for management of lower and upper respiratory tract infections, sinusitis and obstructive lung diseases. The healing herein includes curing, alleviating, reversing, palliative and prophylactic treatment of the condition. The formulations can include total honey or honey extract, such as water, alcohol or ethyl acetate extracts.

The main advantages include;

1—Natural products and available everywhere;

2—Safe and almost no side effects;

3—Synergistic effects as upper and lower respiratory system healer as well as antimicrobial to eradicate infections; and

4—Provides respiratory system with excellent nutritional, anti-inflammatory and antioxidant ingredients.

Another aspect of the invention is to use mixed honey alone or that is mixed with propolis or mixed propolis as an alternative sweet in normal individuals and in patients with diabetes. mellitus. The composition compromises 100% mixed honey or a mixture of 95-99.9% mixed honey and 0.1-5% propolis extract or mixed propolis extract. The recipe might include ingredients that help dissolving propolis in water. The healing herein includes curing, alleviating, reversing, palliative and prophylactic treatment of the condition. The main advantages include;

1—Natural products and available everywhere;

2—Safe and almost no side effects;

3—Better than sucrose or glucose regarding postprandial blood glucose level; and

4—Provides body with excellent nutritional, anti-inflammatory and antioxidant ingredients.

The invention also relates to a vitiligo healing composition, wherein the use of topical mixture of mixed honey, bee venom, nigella sativa or thymoquinone to be used as part of management of vitiligo and depigmented skin and mucous membrane area in form of gel, ointment or cream. The composition applied directly on the affected area 1-4 times a day, or every other day, or weekly with post sun exposure. The recipe might include ingredient that help dissolving propolis in water. The healing herein includes curing, alleviating, reversing, palliative and prophylactic treatment of the condition. The formulae can be used alone or in combination with drugs used in the management of vitiligo. The formulations can include total honey or honey extract, such as water, alcohol or ethyl acetate extracts.

This composition allows batch process production, where it is possible to achieve mixing and fusion of various natural substances, cream homogenization, by converting all substances in dissolvable stage. Hard propolis is converted into dissolvable stage by extraction with ethanol, obtain water extract, or could be used as ground powder. Bee pollen powder is dried and ground into powder. Honey is used as semiliquid. Other ingredients are used as ground powder, or water and alcohol extracts or as an oil form. One or more pharmaceutically acceptable diluents, excipients or carriers can be added. The composition may comprise substances which assist in its application or storage stability, such as, pharmaceutical adjuvant, stabilizers, buffer substances, preservatives, water, thickening agents, emulsifiers, wax, beeswax, and the like. Gels, ointments and creams may be formulated, for example, with an aqueous or oily base with the addition of suitable thickening. Lotions may be formulated with an oily or aqueous base and can also contain one or more emulsifying agents, dispersing agents, stabilizing agents, coloring agents, and/or suspending agents thickening agents.

Suitable carriers also include creams/ointments conventionally used for topical pharmaceutical preparations, such as alginate, preservatives such as benzyl alcohol, buffers to control pH such as disodium hydrogen phosphate/sodium dihydrogen phosphate, agents to adjust osmolarity such as sodium chloride, and stabilizers such as EDTA.

The composition can be applied directly to the wound as a gel, ointment, liquid, cream, or the like as described above. The main advantages includes:

1—Natural products and available everywhere;

2—Safe and almost no side effects;

3—Synergistic effect to stimulate skin pigmentation and melanocytes growth and multiplication;

4—Provides skin with excellent nutritional and anti-inflammatory and antioxidant ingredients.

The intravenous administration of mixed honey or honey extracts with or without propolis extract or mixed propolis extracts or CAPE, thymoquinone, curcumin, or curcumin extract can be used alone or sequentially or simultaneously with the intravenous antibiotics or cytotoxic agents. When administered simultaneously, mixed honey with or without propolis extract or mixed propolis extracts or CAPE can be in the same or separate compositions. Other aspects of the invention relate to the use of mixed honey, propolis, mixed propolis, CAPE, thymoquinone or curcumin as suitable forms for parenteral administration.

The intravenous mixed honey with or without propolis or mixed propolis extracts, or CAPE, thymoquinone or curcumin is for protection of, not limited to, liver, kidney, bone marrow or any other body organs or tissues when used with antibiotics, cytotoxic drugs, and immunosuppressive agents or used in inflammatory diseases, infectious diseases or sepsis.

The amount of the compositions to be administered, the rate of administration and the time of administration will depend on the type of infection to be treated, the antibiotic or cytotoxic agent to be used, and the size, location, progression and/or severity of the infection to be treated. Appropriate dosage for antibiotics or cytotoxic agents, are well known in the art. In some embodiments, mixed honey composition with or without propolis extract or mixed propolis extract is infused daily or every other day or more than one time daily until antibiotic or cytotoxic agent course is finished. Extension of the honey composition infusion might be considered in such case of recurrent infections or malnutrition of severe infection or sepsis. The formulations can include total honey or honey extract, such as water, alcohol or ethyl acetate extracts.

The methods of the present application are useful for the treatment of various infectious diseases, such as bacterial infection, viral infection, fungal infection or parasitic infection, systemic or local.

The compositions may be presented in unit-dose or multi-dose containers. And they may include additional ingredients that are conventional in the art with injectable formulations. The healing herein includes curing, alleviating, reversing, palliative and prophylactic treatment of the condition. Water soluble propolis extract was prepared or using the method described elsewhere (Nikolov N, et al., Bulgarian J Pathology Applied, 1987; 79903/28, 05).

The aims of the composition are:

1—Synergistic effect with antibiotics for treatment of local or systemic infections;

2—For treatment of acute kidney injury and acute liver injury,

3—Hydration,

4—Nutrition supplement, and

5—To decrease the side effects of immunosuppressive agents, cytotoxic agents, or antibiotics.

The invention also relates to a peritonitis healing composition, that comprises mixed honey alone or with propolis or mixed propolis extract or CAPE, curcumin or curcumin extract, nigella sativa oil, and/or thymoquinone in the form of a recipe suitable for parenteral administration. It can be used alone, sequentially, or simultaneously with intravenous antibiotics. When administered simultaneously, the mixed honey with or without propolis extract or mixed propolis extracts or CAPE, or nigella sativa oil, or thymoquinone, or all ingredients together, can be in the same or separate compositions. The composition is used for management of acute or chronic bacterial, viral, fungal or parasitic peritonitis. It could be used in liver cirrhosis. The compositions might include any two of the ingredients Or all the ingredients together. The compositions might be used alone or in combination with antibiotics. Mixed honey can be used alone for the same healing purposes. The formulations can include total honey or honey extract, such as water, alcohol or ethyl acetate extracts.

The amount of the compositions to be administered, the rate of administration and the.time of administration will depend on the type of infection to be treated, progression and/or severity of the infection to be treated. The composition is infused daily or every other day or more than one time daily. Extension of the composition infusion or injection might be considered in such case of recurrent infections or malnutrition or severe infection or sepsis.

The main advantages include;

1—Natural products and available everywhere;

2—Safe and almost no side effects;

3—Synergistic effect as an antimicrobial to eradicate infections; and

4—Provides the peritoneum with excellent nutritional and anti-inflammatory and antioxidant ingredients.

The invention also relates to a herpes simplex 1 and 2 and zoster healing composition that comprises the topical use of mixed honey with or without propolis or mixed propolis (powder or extract) or CAPE, curcumin, bee venom or melittin, cloves or clove extract, garlic or AGE, royal jelly or mixed royal jelly, and nigella sativa or thymoquinone in form of gel, ointment or cream. The mixed honey can be used alone or in combination with one or more than one of the ingredients of the composition. The composition is applied directly on the affected area 3-6 times per day or according to the severity of the lesions. The recipe might include ingredients that help dissolving propolis in water. The healing herein includes curing, alleviating, reversing, palliative and prophylactic treatment of the condition. The composition should be used alone or in combination with drugs used in the management of herpes infection. Multiple compositions can be prepared with mixing any two or more ingredients including mixed honey to achieve the same goals. The formulations can include total honey or honey extract, such as water, alcohol or ethyl acetate extracts.

This composition allows batch process production, where it is possible mixing and fusion of various natural substances, cream homogenization, by converting all substances in dissolvable stage. Hard propolis is converted into the dissolvable stage by extraction with ethanol, is obtained as a water extract or could be used as ground powder. Bee pollen powder is dried and ground into powder. Honey is used as a semiliquid. Other ingredients are used as ground powder, or water and alcohol extracts or as an oil form. One or more pharmaceutically acceptable diluents, excipients or carriers can be added. The composition may comprise substances which assist in its application or storage stability, such as, pharmaceutical adjuvant, stabilizers, buffer substances, preservatives, water, thickening agents, emulsifiers, wax, beeswax, and the like. Gels, ointments and creams may be formulated, for example, with an aqueous or oily base with the addition of suitable thickening. Lotions may be formulated with an oily or aqueous base and can also contain one or more emulsifying agents, dispersing agents, stabilizing agents, coloring agents, and/or suspending agents thickening agents. Suitable carriers also include creams/ointments conventionally used for topical pharmaceutical preparations, such as alginate, preservatives such as benzyl alcohol, buffers to control pH such as disodium hydrogen phosphate/sodium dihydrogen phosphate, agents to adjust osmolarity such as sodium chloride, and stabilizers such as EDTA. The composition can be applied directly to the wound as a gel, ointment, liquid, cream, or the like as described above.

The main advantages include;

1—Natural products and available everywhere;

2—Safe and almost no side effects;

3—Synergistic effects as kidney healer as well as' antimicrobial to eradicate infections; and 4—Provides the urinary system with excellent nutritional and anti-inflammatory and antioxidant ingredients.

The invention also relates to a Clostridia difficile colitis healing composition that comprises the composition of mixed honey alone or with propolis or mixed propolis or CAPE, Nigella sativa or Nigella sativa oil or thymoquinone, curcumin or curcumin extract, cloves or cloves extract, garlic or AGE or garlic oil in form of oral recipe. The composition can be used alone or in combination with antibiotics and with or without an intravenous formula presented in the patent. The compositions may be presented in unit-dose or multi-dose containers. And they may include additional ingredients that are conventional in the art with regard to oral formulations. The healing herein includes curing, alleviating, reversing, palliative and prophylactic treatment of the condition. Multiple compositions can be prepared with mixing any two or more ingredients including honey to achieve the same goals. The compositions can be administered by oral route in dosage unit formulations containing conventional nontoxic pharmaceutically acceptable carriers, adjuvants, and vehicles, as desired. One or more pharmaceutically acceptable diluents, excipients or carriers can be added. The composition may comprise substances which assist in its application or storage stability, such as, pharmaceutical adjuvants, stabilizers, buffer substances, preservatives, water, thickening agents, emulsifiers, beeswax, and the like. The advantageous use of the substance mixture according to the invention consists in preparing the product or composition that will be useful to eradicate clostridia difficile infection. The formulations can include total honey or honey extract, such as water, alcohol or ethyl acetate extracts.

The main advantages include;

1—Natural products and available everywhere;

2—Safe and almost no side effects;

3—Synergistic effects as kidney healer as well as antimicrobial to eradicate infections; and .

4—Provides the urinary system with excellent nutritional and anti-inflammatory and antioxidants ingredients.

The invention also comprises the use of mixed honey alone or a mixture of mixed honey and mixed propolis or propolis are used by oral administration or parenteral administration for management of parasitic diseases. The composition may include other natural ingredients such as garlic, curcumin, nigella sativa, thymoquinone, allicin and CAPE. The composition is used for management of parasitic diseases such as, not limited to, Entamoeba histolytica, Giardia lamella, Blastocystis species, schistosomiasis, malaria, flukes, trypanosome cruzi, hydatid cyst, toxoplasmosis, visceral leishmaniasis, ascaris, nematodes, trematodes, and trichomonas species. The formulations can include total honey or honey extract, such as water, alcohol or ethyl acetate extracts.

The main advantages include;

1—Natural products .and available everywhere;

2—Safe and almost no side effects;

3—Synergistic effects as a kidney healer as well as antimicrobial to eradicate infections; and

4—Provides biological systems with excellent nutritional and anti-inflammatory and antioxidant ingredients.

### DETAILED DESCRIPTION OF THE PREFERRED EMBODIMENTS

The invention is described below with reference to specific experiments and examples.

### Experiment 1

### Antimicrobial Effect of Mixed Honeys

Two types of honeys were collected from different forested geographical areas; both of them were polyfloral in origin. One was light yellow honey and the other one was dark honey. A mixture of both honeys was prepared by mixing light yellow honey with the dark honey in equal parts, and the mixture was stored in a dark place for experimentation. Cultures of various human pathogenic strains were obtained, which included E.coli, S. aureus, and C. albicans. The isolates were identified by the standard bacteriological techniques. Using a 10 μL standard loop, a colony of each isolate was picked from a plate, grown in 10 ml nutrient broth, and used after 24 h incubation in 37° C. Bacterial growth was assessed visually on solid media as: 0: no growth, 1: little growth, 2: mild growth, 3: moderate growth, and 4: heavy growth. Cultures of the specimens of the isolates in broth containing different concentrations of yellow honey, dark honey or mixed honey were performed. The specimen of each microorganism was taken from pure culture grown in the 10 ml nutrient. These cultures were incubated at 37° C. for 24 h. Thereafter, a loopful (10 μl) of the cultures of each of the specimens of microorganisms was streaked onto agar plates to assess the viability of the isolates. The streaked plates were incubated aerobically at 37° C. and inspected after 24 h. Results showed that mixing honey was more potent than yellow honey or dark honey alone to inhibit the growth of the microorganisms tested (Table 1).

TABLE 1
antimicrobial effects of single or mixed honey on human
pathogens.

| Type or | | Con- | Honey concentration % wt/v | | | | | | |
|---|---|---|---|---|---|---|---|---|---|
| honey | Microorganism | trol | 10 | 20 | 30 | 40 | 50 | 60 | MIC |
| Yellow | E. coli | 4+ | 3+ | 3+ | 1+ | 0 | | | 40% |
| honey | S. aureus | 4+ | 3+ | 3+ | 2+ | 1+ | 0 | | 50% |
| | C. albicans | 4+ | 4+ | 3+ | 3+ | 2+ | 1+ | 0 | 60% |
| Dark | E. coli | 4+ | 3+ | 2+ | 1+ | 0 | | | 40% |
| honey | S. aureus | 4+ | 3+ | 3+ | 2+ | 0 | | | 40% |
| | C. albicans | 4+ | 4+ | 3+ | 3+ | 1+ | 1+ | 0 | 60% |
| Mixed | E. coli | 4+ | 2+ | +1 | 0 | | | | 30% |
| honey | S. aureus | 4+ | 2+ | +1 | 0 | | | | 30% |
| | C. albicans | 4+ | 3+ | +2 | +1 | 0 | | | 40% |

MIC = minimum inhibitory concentration.

### Experiment 2

### Effect of Mixed Honey on Wound Healing

20 Swiss mice, 30.8 +/−1.99 g were used for experimentation. Each animal was restrained in a clean and well-ventilated box. The animals were given access to water and feed at libitum. All the experiments were conducted in accordance with the internationally accepted principles for laboratory animal use and care. Under inhalation anesthesia with use of halothane in a drop jar, a 12 mm surgical incision was made on the dorsum of each mouse aseptically. The animals were divided into four groups, each containing four mice. Group one: no treatment, group two: treated with yellow honey applied to wound directly three times a day, group 3: treated with dark honey applied directly to wound three times daily and group four: treated with mixed honey applied directly to the wound three times daily. Assessment of the wound was made by 12 hourly examinations of the wounds for redness, edema, discharge, gapping and closure. Results showed that complete closure was obtained in 7-10 days in control group, in 4-6 days in animals treated with yellow or dark honey and the closure of wound was obtained in 3 days in group treated with mixed honey.

### Experiment 3

### Safety of Intravenous Mixed Honey Infusion

This experiment was carried out to investigate whether intravenous mixed honey could elicit immediate or remote adverse effects. Two multifloral honeys (honey A and honey B) were collected from different geographical areas, and were mixed together in ratio 1:1 wt/wt. All the experiments were conducted in accordance with the internationally accepted principles for laboratory animal use and care.

Nine sheep were used in this experiment. Blood investigations, including hematological and biochemical tests, were performed after 12 h of fasting. Then the animals received intravenous honey A solution, in a dose of 1 gram per kg.b.wt., intravenous honey B solution, in a dose of 1 gram per kg.b.wt, and intravenous mixed honey solution, in a dose of 1 gram per kg.b.wt.

Each honey sample was dissolved in 300 mL of normal saline (about a 10% honey solution). The infusion was given over a three hours. During infusion, animals were observed for any abnormal signs such as shortness of breath, shivering, allergic reaction, restlessness, or changes in the level of consciousness. Blood investigations were repeated after 5 days. When the animals showed no reaction to the first dose of honey infusion, they received similar doses of the honey solutions by intravenous rout at same doses after 30 days. Blood investigations were repeated 5 days after the second infusion. The animals were kept for 3 months of observation of adverse effects.

The results showed that no adverse effects were obtained during and after mixed honey infusion and all the sheep received the second infusion of mixed honey solution without harmful effects. No significant changes in blood urea nitrogen (BUN), serum creatinine, liver enzymes (SGPT, SGOT), blood sugar, white blood cell or hemoglobin were observed after mixed honey infusion (Table 2).

TABLE 2
effect of intravenous administration of single honey A and
B and mixed (A + B) honey on blood variables

| | Honey A | | | Honey B | | | Mixed honey | | |
|---|---|---|---|---|---|---|---|---|---|
| | | First | Second | | | Second | | | Second |
| Variables | baseline | dose | dose | baseline | First dose | dose | baseline | First dose | dose |
| BUN | 16.4 +/− 2.4 | 15.7 +/− 4.2 | 16 +/− 5.1 | 18 +/− 5.1 | 17.8 +/− 6 | 17 +/− 3 | 15.1 +/− 4 | 13.7 +/− 2 | 13 +/− 2.2 |
| Creatinine | 1.3 +/− 0.3 | 1.1 +/− 0.1 | 1.0 +/− 0.2 | 1.2 +/− 0.4 | 1.1 +/ 0.45 | 0.9 +/− 0.33 | 1.25 +/− 0.23 | 1 +/ 0.4 | 0.89 +/− 0.3 |
| SGOT | 40 +/− 6.8 | 38.8 +/− 7 | 36 +/− 5.1 | 36 +/− 4.4 | 37.1 +/− 2.1 | 36 +/− 3.5 | 39 +/− 2.2 | 38.1 +/ 3.2 | 35.6 +/− 3.6 |
| SGPT | 20 +/− 4.5 | 18.9 +/− 3.3 | 17.8 +/− 5.1 | 19 +/− 5.2 | 19 +/− 4.1 | 17.8 +/− 3.0 | 21 +/− 2.5 | 19.2 + −4.2 | 17 +/− 5.5 |
| WBC | 9.5 +/− 1.2 | 10.1 +/− 2.1 | 10.4 +/− 1.9 | 11.1 +/− 1.1 | 11 +/− 2.1 | 11.5 +/− 3.4 | 10.8 +/− 4.2 | 11.2 +/− 3.2 | 11.7 +/− 2.8 |
| hemoglobin | 12.1 +/− 1.2 | 12.0 +/− 2.2 | 13.2 +/− 1.8 | 11.4 +/− 2.4 | 10.8 +/− 3.2 | 11.4 +/− 3.4 | 10.2 +/− 2.6 | 11 +/2 | 12.5 +/− 1.8 |
| Blood sugar | 121 +/− 10 | 122 +/− 12 | 110 +/− 14 | 125 +/− 11 | 120 +/− 15 | 118 +/− 9 | 123 +/− 13 | 118 +/− 14 | 110 +/− 12.5 |

### Experiment 4

### Mixture of Honey/Propolis in Parasitic Infection

Fifteen patients with Entamoeba histolytica, infection (trophozoites and cysts) where divided into three groups, group 1 (5 patients) treated with oral honey-1 g/kg.b.wt daily, group 2 (five patients) treated with propolis 0.1 mg/kg.b.wt daily, and group 3 (5 patients) treated with honey/propolis mixture. Stool examination showed no trophozoites or cysts by day 4-6 in all the patients treated with honey/propolis mixture and by 5-7 days in 3/5 patients treated by either honey or propolis. Thus adding propolis to the honey increases its effectiveness in fighting parasitic infections.

The same experiments were repeated in other 15 patients with Giardia lamella infection and 15 patients with Trichomonas vaginalis infection. The results showed honey/propolis mixture was more effective than honey or propolis alone.

### Experiment 5

### Mixed Honey in Parasitic Infection

Fifteen patients with Entamoeba histolytica infections were divided into three treatment groups. Group 1 (5 patients) treated with honey A (1 g/kg.b.wt) per day; group 2 (5 patients) treated with honey B (1 g/kg.b.wt) per day; and group three (5 patients) were treated with mixed honey (A+B) in similar dose (1 g/kg.b.wt) daily. In first and second group 3/5 patients showed no infection by 5-7 days of treatment and 5/5 patients in group three showed no infection in 5-6 days. The same experiments were repeated in other 15 patients with Giardia lamella infection and 15 patients with Trichomonas vaginalis infection. The results showed that mixed honey was more effective than honey A or honey B alone.

The tables below provide suggested compositions of mixed honey along with other ingredients for treating various conditions.

TABLE 3
Honey based wound healing composition. It includes mixed honey or mixed honey extract alone
or all the ingredients mixed together, or mixed honey is mixed with any one of the ingredients
or mixed with two or more than two of the ingredients. One or more pharmaceutically acceptable
diluents or carriers can be added to adjust the dose such as beeswax. Any two or more than
two of the ingredients can be mixed together to prepare skin/wound healing compositions not
including honey or propolis. The compositions might include acacia Arabica 1-70%,
clove or clove oil 1-70%, garlic or aged garlic or AGE or garlic oil 1-50%, curcumin 1-80%,
olive oil 1-50%, nigella sativa, thymoquinone 1-60% wt/wt, bee pollen or mixed
bee pollen 4-20%, royal jelly or mixed royal jelly, 5-20%.

| | Concentrations | | | |
|---|---|---|---|---|
| | | Ethanol or water | | |
| | | extract or ethyl | | Example of |
| Ingredients | Crude or Powder | acetate extract | oil | formulae |
| Mixed honey | 10-99% | 5-49% | | 50% |
| Propolis or mixed propolis | 1-30% | 1-15% | | 10% |
| Or CAPE | 0.1-1000 | | | 0r CAPE 50 |
| | micromole/100 g | | | micromole/100 g |
| Nigella sativa | 0.1-15% | 0.1-15% | 0.1-10% | 5% |
| or Thymoquinone | 0.1-5% | | | Or 5% |
| | | | | thymoquinone |
| Curcumin | 5-20% | 5-10% | | 5% |
| Garlic crude or | 5-20% | 1-10% | 0.5-3% | 5% |
| Aged Garlic Extract | 1-15% | 1-5% | | Or 5% aged |
| | | | | garlic extract |
| Olive oil | 5-10% | | | 6% |
| Bee pollen powder | 4-10% | 3-6% | | 5% |
| or mixed bee pollen | | | | |
| Clove | 1-15% | 0.5-10% | 0.1-10% | 4% |
| Bee venom | 0.1-15 mg/100 g | 0.1-15 mg/100 g | | 0.1 mg/100 g |
| or Melittin | 0.1-5 mg/100 g | 0.1-5 mg/100 g | | or melittin |
| | | | | 0.1 mg/100 g |
| Acacia Arabica | 1-20% | 1-10% | | 4% |
| Royal jelly or mixed | 1-20% | 5-10% | | 6% |
| royal jelly | | | | |

TABLE 4
Wart healing composition It includes mixed honey or mixed honey extract alone or all the
ingredients mixed together, or honey is mixed with any one of the ingredients or mixed
with two or more than two of the ingredients, or any two or more than two of the ingredients
are mixed together without honey or propolis. One or more pharmaceutically acceptable diluents
or carriers can be added to adjust the dose such as beeswax. Multiple compositions can be
prepared with mixing two or more ingredients not including honey. The compositions might
include clove or clove oil 1-70%, garlic or aged garlic or AGE or garlic oil 1-50%, olive oil
1-50%, and nigella sativa or thymoquinone 1-60% wt/wt.

| | Concentrations | | | |
|---|---|---|---|---|
| | | Ethanol or water | | |
| | | or ethyl acetate | | Example of |
| Ingredients | Crude or Powder | extract | oil | formulae |
| Mixed honey | 30-70% | | | 40% |
| Propolis or mixed propolis | 1-40% | 1-30% | | 30% |
| Or CAPE | 1-100 | | | Or CAPE 50 |
| | micromole/100 g | | | micromole/100 g |
| Nigella sativa | 0.1-20% | 0.1-20% | 0.1-10% | 10% |
| or Thymoquinone | 0.1-10% | | | Or 10% |
| | | | | thymoquinone |
| Garlic crude | 5-20% | 1-10% | 0.5-3% | 5% |
| or Aged Garlic Extract | 1-15% | 1-5% | | Or 5% aged |
| | | | | garlic extract |
| Olive oil | 5-10% | | | 5% |
| Cloves | 1-15% | 0.5-15% | 0.1-15% | 15% |

TABLE 5
Cutaneous leishmaniasis healing composition. It includes mixed honey or mixed honey extract
alone or all the ingredients mixed together, or honey is mixed with any one of the ingredient
or mixed with two or more than two of the ingredient. One or more pharmaceutically acceptable
diluents or carriers can be added to adjust the dose such as bee wax. Multiple compositions can
be prepared with mixing two or more ingredients not including honey and propolis. The
compositions might include clove or clove oil 1-70%, garlic or aged, garlic or AGE or garlic oil
1-50%, curcumin 1-80%, and nigella sativa or thymoquinone 1-60% wt/wt.

| | Concentrations | | | |
|---|---|---|---|---|
| | | Ethanol or water | | |
| | | or ethyl acetate | | Example of |
| Ingredients | Crude or Powder | extract | oil | formulae |
| Mixed honey | 30-70% | | | 50% |
| Propolis or mixed propolis | 1-30% | 1-20% | | 20% |
| or CAPE | 1-1000 | | | Or CAPE 50 |
| | micromole/100 g | | | micromole/100 g |
| Nigella sativa | 0.1-20% | 0.1-10% | 0.1-10% | 10% |
| or Thymoquinone | 0.1-10% | | | Or 10% |
| | | | | thymoquinone |
| Curcumin | 5-20% | 5-10% | | 5% |
| Garlic crude | 5-20% | 1-15% | 0.5-3% | 10% |
| or Aged Garlic Extract | 1-15% | 1-10% | | Or 10% aged |
| | | | | garlic extract |
| Cloves | 1-15% | 0.5-10% | 0.1-10% | 5% |

To one skilled in the art, various modifications of the invention, without departing from the scope may include:
1—incorporating new components to optimize efficacy or commercial processing
2—altering the % weight of each constituent
3—introducing isolated chemical compounds derived from each ingredient extract individually or in combination to produce similar or better therapeutic results
4—or changing the formulation by subtracting aforementioned ingredients or using various combinations.

All of the ingredients are natural substances and have various antimicrobial, antioxidant, anti-inflammatory and nutritional properties.

### Intraperitoneal Infection

Intra-abdominal infection and peritonitis are an important cause of morbidity and mortality. It is the second most commonly identified cause of severe sepsis in the intensive care unit. Peritonitis might be a result of perforated appendicitis, perforation of peptic ulcer disease, and diverticulitis with perforation, or due to intestinal perforation due to post-traumatic, post-operative, or post-interventional. Intra-abdominal infection or intraperitoneal includes peritoneal inflammation in response to infection with microorganisms, resulting in purulence in the peritoneal cavity. The infection might involve intramural inflammation of the gastrointestinal and extended into the peritoneal space. They cause peritoneal inflammation, and are associated with localized or diffuse peritonitis. Primary peritonitis or spontaneous bacterial peritonitis result of bacterial translocation across an intact gut wall as in. liver cirrhosis. Secondary peritonitis is caused by a gut perforation, laceration, or necrotic segment of the tract. Early diagnosis and management with broad-spectrum antibiotics are essential. The patent presents new composition with natural ingredients that provides excellent antimicrobial, antioxidants and anti-inflammatory intervention. A peritonitis healing composition can be created using the table below:

TABLE 6
Peritonitis healing composition. It includes mixed honey or mixed
honey extract alone or all the ingredients mixed together, or
honey is mixed with any one of the ingredients or mixed with
two or more than two of the ingredients. Multiple compositions
including any two or more than two of the ingredients can be
prepared without honey or propolis. One or more pharmaceutically
acceptable diluents or carriers can be added to adjust the dose
and to make the administration of the ingredients parenterally
feasible. Daily dose of the formulae 0.5-3 gram/kg.b.wt.

| | Doses | | Example of | |
|---|---|---|---|---|
| Ingredients | (per kg/day) | | the formulae | |
| Mixed honey or | 1-2 | g | 70 | g |
| Honey extract in water, | .5-1 | g | | |
| ethanol or ethyl acetate | | | | |
| Curcumin extract | 0.001-0.1 | g | 1 | g |
| Thymoquinone | 0.002-0.02 | g | 0.5 | g |
| CAPE | 0.001-0.03 | g | 0.5 | g |
| Propolis or mixed propolis extract | 0.005-0.1 | g | 1 | g |
| Nigella sativa extract | 0.005-0.1 | g | 1 | g |
| Or Nigella sativa oil | 0.0142-0.5 | ml | 5 | ml |

Intraperitoneal honey or mixed honey with propolis or mixed propolis extract, CAPE, curcumin, nigella sativa oil or thymoquinone are used with or without antibiotics. The compositions may be presented in unit-dose or multi-dose containers.

Honey and propolis have a potent antimicrobial activity. Therefore, the co-administration of honey and propolis intraperitoneal, intravenously or orally together might potentiate their antimicrobial effect and would be useful for treatment of peritonitis and prevention of peritoneal adhesion.

### Intravenous use of Honey and Propolis for Infection, Dehydration and Nutrition

Dehydration and malnutrition are common in clinical practice. Parenteral nutrition with lipids, protein and various compound are used. For hydration, various concentrations of dextrose, potassium and sodium chloride are used. The patent presents new compositions for management of local or systemic infections (due to bacteria, parasites or viruses), sepsis, dehydration, and malnutrition or as part of nutritional support with strong antioxidants, antimicrobial and anti-inflammatory properties. The invention will be illustrated below with the following example, which is in no way restrictive.
Mixed honey or water, ethanol or ethyl acetate extract : 0.1-2 g/k.b.wt
Propolis or mixed propolis extract: 0.1-30 mg/kg.b.wt
CAPE: 0.001-10 mg/kg.b.wt or 0.1-500 micromole/kg.b.wt
Thymoquinone: 0.001-5 mg/kg.b.wt
Curcumin or curcumin extract: 0.1-40 mg/kg

The dose is given 1-4 times daily or every other day, or weekly or according to the state and severity of dehydration, infection or malnutrition. Mixed honey alone or mixed with one or more than one of the ingredients can be used as part of management of malignancies and to potentiate the potency of anti-cancer therapy such as radiotherapy and chemotherapy and to heal their side effects.

To one skilled in the art, various modifications of the invention, without departing from the scope may include:
1—incorporating new components to optimize efficacy or commercial processing
2—altering the percent weight of each constituent
3—introducing isolated chemical compounds derived from each ingredient extract individually or in combination to produce similar or better therapeutic results
4—or changing the formulation by subtracting aforementioned ingredients or using various combinations.

The compositions are used alone or in combination with antibiotics. Some of the individual ingredients presented in the compositions were tested in bacterial infections but the compositions that included mixture of the ingredients such as illustrated in the patent are not published. or tested in bacterial, viral or parasitic infections, dehydration, or for nutritional purposes. Furthermore, intravenous mixed honey or mixed propolis were never tested in bacterial, viral or parasitic local or systemic infections or malignancies.

### Herpes Infection

Herpes simplex virus type 1 is among the most prevalent infectious pathogens. It can cause fatal disseminated disease in newborns, cold sores, eye disease, and fatal encephalitis in adults. Genital herpes is a common sexually transmitted disease caused by herpes simplex type 1 and type 2 and characterized by lifelong infection and periodic reactivation.

The patent presents multiple compositions with antiviral, antimicrobial, anti-inflammatory and anti-oxidant activities. The invention will be illustrated below with the following examples, which are in no way restrictive.

TABLE 7
Herpes healing composition. It includes mixed honey or mixed honey extract alone or
all the ingredients mixed together, or honey is mixed with any one of the ingredients
or mixed with two or more than two of the ingredients, or two or more than two of the
ingredients are mixed together not including honey. One or more pharmaceutically acceptable
diluents or carriers can be added to adjust the dose such as beeswax.

| | Concentrations | | | |
|---|---|---|---|---|
| | | Ethanol or water | | |
| | | or ethyl acetate | | Example of |
| Ingredients | Crude or Powder | extract | oil | formulae |
| Mixed honey | 30-99% | 15-49% | | 60% |
| Propolis or mixed propolis | 1-30% | 1-15% | | 10% |
| Or CAPE | 1-100 | | | Or CAPE 50 |
| | micromole/100 g | | | micromole/100 g |
| Nigella sativa | 0.1-5% | 0.1-5% | | 5% |
| Or Thymoquinone | | | | |
| Curcumin | 5-20% | 5-10% | | 10% |
| Cloves | 1-15% | 0.5-10% | 0.1-10% | 5% |
| Bee venom | 0.1-15 mg/100 g | 0.1-15 mg/100 g | | 0.2 mg/100 g |
| or Melittin | 0.1-5 mg/100 g | 0.1-5 mg/100 g | | or melittin |
| | | | | 0.1 mg/100 g |
| Royal jelly or mixed royal | 1-20% | 5-10% | | 6% |
| jelly | | | | |
| Garlic or AGE | 5-20% | 1-10% | 0.5-3% | 5% |
| | or AGE 1-15% | | | |

To one skilled in the art, various modifications of the invention, without departing from the scope may include:
1—incorporating new components to optimize efficacy or commercial processing
2—altering the percent weight of each constituent
3—introducing isolated chemical compounds derived from each ingredient extract individually or in combination to produce similar or better therapeutic results
4—or changing the formulation by subtracting aforementioned ingredients or using various combinations.

Some of the individual ingredients presented in the compositions was tested in herpes simplex infections but the compositions that included mixture of the ingredients such as illustrated in the patent are not published or tested in herpes simplex type 1 and 2 infection or herpes zoster infection. Furthermore, mixed honey or mixed propolis were never tested in herpes simplex type 1 and 2 infection or herpes zoster infection.

### Chronic Diseases and General Oral Composition

Chronic disease such as diabetes mellitus, hypertension, cardiovascular diseases, dyslipidemia and immunological diseases are common. There is no cure for any of them, but chronic treatment is necessary to reduce their complications. Nutritional factors, inflammatory mediators and oxidative stress play a major role in the pathogenesis. The oral general healing composition offers the advantage of using ingredients of a natural origin which are readily available and whose association shows excellent high nutritional value, antioxidant and inflammatory property, and antimicrobial activity and anti-tumor activity. The composition of the invention also offers the advantage of being easy to intake orally.

The invention is intended for the management of chronic disease solely or as part of global management, that includes changed lifestyle and modern interventions. The compositions may be presented in unit-dose or multi-dose containers. And they may include additional ingredients that are conventional in the art with oral formulations. The composition of the invention may be in different galenical forms such as in liquid, semisolid, gel, capsule, or tablet. The invention will be illustrated below with the following examples, which are in no way restrictive.

TABLE 8
General oral healing formulae. It includes mixed honey or mixed honey extract
alone or all the ingredients mixed together, or honey is mixed with any one of
the ingredients or mixed with two or more than two of the ingredients. One or
more pharmaceutically acceptable diluents or carriers can be added to adjust the
dose. Daily dose of the formulae 0.5-3 gram/kg.b.wt. Two or more than two ingredients
can be mixed together to form multiple oral formulations not including honey or
propolis. Other ingredients of the mixtures might include date seed powder (5-
20%), why protein (5-20%), peanuts or any other type of nuts (5-20).

| | Doses per 70 kg.b.wt/day | | |
|---|---|---|---|
| | | | Example of |
| | Gram/kg.b.wt/ | | the formulae |
| Ingredients | day | Gram/day | gram |
| Mixed honey | 0.1-2 | 7-140 | 50 |
| or Honey extract with water, | 0.5-1 | 3.5-70 | 25 |
| ethanol or ethyl acetate | | | |
| Royal jelly or mixed royal jelly | 0.014-0.85 | 1-6 | 5 |
| Nigella sativa | 0.007-0.28 | 0.5-20 | 5 |
| Or Thymoquinone | 0.001-0.4 | 0.07-2.8 | 1 |
| Or Nigella sativa oil | 0.014-0.14 | 0.1-10 | 3 |
| Acacia Arabica | 0.07-0.42 | 1-30 | 10 |
| Garlic | 0.014-0.285 | 1-20 | 5 |
| Or aged garlic extract | 0.014-0.85 | 1-6 | 5 |
| Or garlic oil | 0.0014-0.071 | 0.1-5 | 0.5 |
| Propolis or mixed propolis | 0.011-0.285 | 0.8-20 | 5 |
| Or propolis extract | 0.005-0.21 | 0.4-15 | 5 |
| Or CAPE | 0.001-003 | 0.07-0.21 | 0.1 |
| Bee pollen or mixed bee pollen | 0.142-0.57 | 10-40 | 15 |
| Olive oil | 0.142-0.57 | 10-40 | 20 |
| Curcumin or curcumin extract | 0.007-0.42 | 0.5-20 | 10 |
| Cloves or cloves extract | 0.007-0.14 | 0.5-10 | 3 |
| Or clove oil | 0.014-0.14 | 1-10 | or clove oil 2 ml |
| Flaxseed | 0.142-0.57 | 10-40 | 10 |
| Or flaxseed oil | 0.014-0.085 | 1-6 | 1 |
| Green tea or green tea extract | 0.007-0.071 | 0.5-5 | 1 |
| Fish oil | 0.014-0.042 | 1-3 | 2 |
| Walnuts | 0.011-0.71 | 20-50 | 20 |
| Or walnuts oil | 0.014-0.071 | Or oil 1-5 | Or oil 3 |
| Ginger powder | 0.014-0.285 | 1-20 | 5 |
| Or Ginger water extract | 0.014-0.285 | 1-20 | Or water extract 3 |
| Or Ginger ethanol extract | 0.0014-0.15 | 0.1-10 | Or ethanol extract 2 |
| Fenugreek seeds powder | 0.028-0.214 | 2-15 | 5 |
| Or extract | 0.004-0.028 | 0.3-2 | Or extract 1 |

To one skilled in the art, various modifications of the invention, without departing from the scope may include:
1—incorporating new components to optimize efficacy or commercial processing,
2—altering the % weight of each constituent,
3—introducing isolated chemical compounds derived from each ingredient extract individually or in combination to produce similar or better therapeutic results, or
4—changing the formulation by subtracting aforementioned ingredients or using various combinations.

Some of the individual ingredients presented in the compositions were tested in the chronic diseases but the compositions that included mixture of the ingredients such as illustrated in the patent are not published or tested in the chronic or acute diseases intended to be treated by the compositions. Furthermore, oral mixed honey or mixed propolis, mixed bee pollen and mixed royal jelly were never tested in management of acute or chronic disease.

Honey is the major ingredient in the oral healing formulae. Others and we have found that honey is a rich source of natural nutrients, and suitable to be used in normal subjects and in patients with diabetes mellitus, hypertension, chronic kidney disease and cardiovascular disease.

H. pylori Infection

It was estimated that H. pylori infects more than half of the human population worldwide. It causes chronic active gastritis, peptic ulcer disease and gastric cancer. It is involved in the development of peptic ulcer disease in 10%-20%, and gastric lymphoma in 1%-2% of the infected individuals.

The first-line treatment consists of two antimicrobials, amoxicillin with clarithromycin or metronidazole, plus a proton-pump inhibitor. A quadruple second-line therapy is recommended when other intervention is failed, consisting of a combination of bismuth salts, tetracycline and metronidazole and a proton-pump inhibitor. The major problem is a bacterial antibiotic resistance and a protective environment of stomach mucus layer and the epithelial cells. Therefore, it is necessary to find new effective agents.

The patent presents various compositions with antimicrobial, anti-inflammatory and antioxidant activity for treatment of H. pylori—induced diseases and for eradication of the infection. The invention will be illustrated below with the following examples, which are in no way restrictive.

TABLE 9
H. pylori composition includes mixed honey or mixed honey extract alone or all
the ingredients mixed together, or honey is mixed with any one of the ingredients
or mixed with two or more than two of the ingredients. One or more pharmaceutically
acceptable diluents or carriers can be added to adjust the dose. Daily dose of the
composition 0.5-3 gram/kg.b.wt. Multiple compositions can be prepared by mixing
two more than two of the ingredients not including honey or propolis.

| | Doses per 70 kg.b.wt/day | | |
|---|---|---|---|
| | | | Example of |
| | Gram/ | | the formulae/ |
| Ingredients | kg.b.wt/day | Gram/day | gram |
| Mixed honey | 0.1-2 | 7-140 | 50 |
| or Extract with water, | .05-1 | | |
| ethanol or ethyl acetate | | | |
| Nigella sativa | 0.014-0.28 | 1-20 | 5 |
| Or Thymoquinone | 0.001-0.4 | 0.07-2.8 | 1 |
| Or Nigella sativa oil | 0.014-0.14 | 0.1-10 | 3 |
| Acacia Arabica | 0.07-0.42 | 1-30 | 10 |
| Garlic | 0.014-0.285 | 1-20 | 5 |
| Or aged garlic extract | 0.014-0.85 | 1-6 | 5 |
| Or garlic oil | 0.0014-0.071 | 0.1-5 | 0.5 |
| Propolis or mixed propolis | 0.011-0.285 | 0.8-20 | 5 |
| Or propolis extract | 0.005-0.21 | 0.4-15 | 5 |
| Or CAPE | 0.001-003 | 0.07-0.21 | 0.1 |
| Bee pollen or mixed bee pollen | 0.142-0.57 | 10-40 | 15 |
| Olive oil | 0.142-0.57 | 100-40 | 20 |
| Curcumin or curcumin extract | 0.014-0.42 | 1-20 | 10 |
| Cloves or cloves extract | 0.007-0.14 | 0.5-10 | 1 |
| Or clove oil | 0.014-0.14 ml | 1-10 ml | Or clove oil 2 ml |
| Fish oil | 0.014-0.042 | 1-3 | 2 |

To one skilled in the art, various modifications of the invention, without departing from the scope may include:
1—incorporating new components to optimize efficacy or commercial processing
2—altering the % weight of each constituent
3—introducing isolated chemical compounds derived from each ingredient extract individually or in combination to produce similar or better therapeutic results
4—or changing the formulation by subtracting aforementioned ingredients or using various combinations.
   Some of the individual ingredients presented in the compositions were tested in H. pylori infection but the compositions that included mixture of the ingredients such as illustrated in the patent are not published or tested in the H. pylori infection or dyspepsia. Furthermore, oral mixed honey or mixed propolis, mixed bee pollen were never tested in management of H.pylori.

### Inhalation Composition for Respiratory Diseases

Mixed honey (comprises from 10-70% w/v mixed honey in suitable solution such as normal saline) is mixed with propolis or mixed propolis extract (comprises from 0.1-30% wt/v propolis or mixed propolis extract in suitable solution such as normal saline). The mixture can include any ratio, preferably 1:1 or 2:1 (honey/propolis). The dose could be changed to any other doses found to be more effective with lowest or no side effects. The composition offers the advantage of using ingredients of a natural origin which are readily. available and whose association shows excellent high nutritional value, bronchodilator activity, wound healing property, and antioxidant, anti-inflammatory action and antimicrobial activity. Mixed honey can be used alone without propolis.

The composition of the invention also offers the advantage of being easy to use with a nebulizer machine, and the patient inhales the ingredient 1-3 times a day. The invention is intended for management of lower and upper respiratory tract infections, sinusitis and obstructive lung diseases and can be used alone or with conventional intervention and with or without oral healing formulae. The compositions may be presented in unit-dose or multi-dose containers. And they may include additional ingredients that are conventional in the art with inhalation formulations. The composition of the invention may be in different galenical forms such as in solution form. Various modifications of the composition without departing from the scope may include incorporating new components to optimize efficacy or commercial processing, altering the percent weight of each constituent, introducing isolated chemical compounds derived from each ingredient individually or in combination to produce similar or better therapeutic results, or changing the formulation by subtracting aforementioned ingredients or using various combinations.

Some of the individual ingredients presented in the compositions were tested as inhalers to the respiratory system but the compositions that included mixture of the ingredients such as illustrated in the patent are not published or tested in upper or lower respiratory diseases. Furthermore, inhaled mixed honey or mixed propolis were never tested in management of respiratory diseases.

### Kidney and Urinary System Diseases

Chronic kidney diseases are common and ultimately lead to end stage renal disease and dialysis. No effective intervention is available at the present time that is capable to stop kidney deterioration. The most common cause of kidney damage is diabetes, hypertension, inflammatory chronic entities, calculus and infection.

The renal healing composition according to the invention offers the advantage of using ingredients of a natural origin which are readily available and whose association shows excellent high nutritional value, renal protection property, potent antioxidant activity, anti-inflammatory action, diuretic activity, urinary nitric oxide stimulation, and antimicrobial activity. The composition can also ameliorate proteinuria. The composition of the invention also offers the advantage of being easy to use as an oral recipe. The invention is intended for the treatment of proteinuria, urinary crystaluria, urinary calculus, and acute and chronic kidney failure due to, but not limited to, diabetic nephropathy, autoimmune disorders, cytotoxic or immunosuppressive agents, or infectious causes. The compositions may be presented in unit-dose or multi-dose containers. And they may include additional ingredients that are conventional in the art with oral formulations. The composition of the invention may be in different galenical forms gel, liquid, or semiliquid.

The invention will be illustrated below with the following examples, which are in no way restrictive.

TABLE 10
Kidney composition. It includes mixed honey or mixed honey extract alone or all
the ingredients mixed together, or honey is mixed with any one of the ingredients
or mixed with two or more than two of the ingredients. One or more pharmaceutically
acceptable diluents or carriers can be added to adjust the dose. Daily dose of the
composition 0.5-3 gram/kg.b.wt. Two or more than two ingredients can be mixed
together to form new compositions without using mixed honey and propolis.

| | Doses | | |
|---|---|---|---|
| | | | Example of |
| Ingredients | Gram/kg.b.wt | Gram/day | the formulae |
| Mixed honey | 0.1-2 | 7-140 | 50 |
| or Extract with water, | .05-1 | 3.5-70 | 25 |
| alcohol or ethyl acetate | | | |
| Royal jelly or mixed royal jelly | 0.014-0.085 | 1-6 | 5 |
| Nigella sativa | 0.014-0.28 | 1-20 | 10 |
| Or Thymoquinone | 0.001-0.4 | 0.07-2.8 | 1 |
| Or Nigella sativa oil | 0.014-0.14 | 0.1-10 | 5 |
| Acacia Arabica | 0.07-0.42 | 1-30 | 10 |
| Garlic | 0.014-0.285 | 1-20 | 5 |
| Or aged garlic extract | 0.014-0.14 | 1-10 | 5 |
| Or garlic oil | 0.0014-0.071 | 0.1-5 | 0.5 |
| Propolis or mixed propolis | 0.011-0.141 | 0.8-10 | 5 |
| Or propolis extract | 0.005-0.141 | 0.4-10 | 5 |
| Or CAPE | 0.001-003 | 0.07-0.21 | 0.1 |
| Bee pollen or mixed bee pollen | 0.142-0.57 | 10-40 | 20 |
| Olive oil | 0.142-0.57 | 100-40 | 30 |
| Curcumin or curcumin extract | 0.014-0.42 | 1-30 | 20 |
| Flaxseed | 0.142-0.57 | 10-40 | 10 |
| Or flaxseed oil | 0.014-0.085 | 1-6 | 1 |
| Ginger powder | 0.014-0.285 | 1-20 | 5 |
| Or ginger water extract | 0.014-0.285 | 1-20 | Or water extract 3 |
| Or ginger ethanol extract | 0.0014-0.15 | 0.1-10 | Or ethanol extract 2 |
| Green tea or green tea extract | 0.0071-0.071 | 0.5-5 | 1 |

Various modifications of the composition without departing from the scope may include incorporating new components to optimize efficacy or commercial processing, altering the percent weight of each constituent, introducing isolated chemical compounds derived from each ingredient individually or in combination to produce similar or better therapeutic results, or changing the formulation by subtracting aforementioned ingredients or using various combinations.

Some of the individual ingredients presented in the compositions were tested in kidney diseases but the compositions that included mixture of the ingredients such as illustrated in the patent are not published or tested in the acute or chronic kidney diseases. Furthermore, oral mixed honey or mixed propolis, or mixed bee pollen or mixed royal jelly were never tested in management of kidney diseases.

### Clostridium Difficile Colitis

Clostridium difficile is a Gram positive anaerobic spore-forming bacillus. In less than 5% of humans, it is part of normal flora. However, infection with Clostridium difficile is a serious problem and it accounts for 30-50% of hospital acquired infections. The bacteria have caused severe infections, diarrhea, pseudomembranous colitis, toxic megacolon, perforation of the colon, and sepsis. It is commonly caused by antibiotic use, which suppresses normal flora of the gut and allows the proliferation of Clostridium difficile with the production of toxins (TcdA and TcdB). The invention will be illustrated below with the following examples, which are in no way restrictive.

TABLE 11
Clostridium difficile colitis composition. It includes mixed
honey or mixed honey extract alone or all the ingredients mixed
together,, or mixed honey is mixed with any one of the ingredients
or mixed with two or more than two of the ingredients. One or more
pharmaceutically acceptable diluents or carriers can be added to
adjust the dose. Daily dose of the formulae 0.5-3 gram/kg.b.wt.
More compositions could be prepared by mixing two or more than two
of the ingredients not including honey and propolis.

| | Doses per 70 kg.b.wt/day | | |
|---|---|---|---|
| | | | Example of |
| | Gram/ | | the formulae |
| Ingredients | kg.b.wt/day | Gram/day | gram |
| Mixed honey | 0.1-2 | 7-140 | 50 |
| or extract with water, | .05-1 | 3.5-70 | 25 |
| alcohol or ethyl acetate | | | |
| Nigella sativa | 0.014-0.28 | 1-20 | 10 |
| Or Thymoquinone | 0.001-0.4 | 0.07-2.8 | 1 |
| Or Nigella sativa oil | 0.014-0.14 | 0.1-10 | 5 |
| Garlic | 0.014-0.285 | 1-20 | 5 |
| Or aged garlic extract | 0.014-0.85 | 1-6 | 5 |
| Or garlic oil | 0.0014-0.071 | 0.1-5 | 0.5 |
| Propolis or mixed | 0.011-0.285 | 0.8-20 | 5 |
| propolis | | | |
| Or propolis extract | 0.005-0.21 | 0.4-15 | 5 |
| Or CAPE | 0.001-003 | 0.07-0.21 | 0.1 |
| Curcumin or curcumin | 0.014-0.42 | 1-30 | 20 |
| extract | | | |
| Cloves or cloves extract | 0.007-0.14 | 0.5-10 | 3 |

To one skilled in the art, various modifications of the invention, without departing from the scope may include:
1—incorporating new components to optimize efficacy or commercial processing
2—altering the % weight of each constituent
3—introducing isolated chemical compounds derived from each ingredient extract individually or in combination to produce similar or better therapeutic results
4—or changing the formulation by subtracting aforementioned ingredients or using various combinations.
   The compositions can be used alone for management of Clostridia difficile colitis or in combination with standard antibiotics. The formulae can be used for acute attack or recurrent attack of Clostridia difficile colitis. The compositions may be presented in unit-dose or multi-dose containers. The composition offers the advantage of using ingredients of a natural origin which are readily available and whose association shows excellent high nutritional value, wound healing property, and antioxidant, anti-inflammatory and antimicrobial activities.

### Probiotics—Gut Bacteria

The gut microbiota are important for vitamin biosynthesis, bile acid degradation, carbohydrate digestion, contribution of nutrients, and regulation of intestinal hormones. Gut microbiota play an important role in human diseases, such as obesity, fatty liver disease, insulin resistance/diabetes, and cardiovascular events.

The gut microbiota enhancing composition offers the advantage of using ingredients of a natural origin which are readily available and whose association shows excellent high nutritional value, antioxidant, and inflammatory action. The composition of the invention also offers the advantage of being easy to ingest and being safe.

The invention is intended to: 1—maintain and enhance the gut microbiota in normal individuals and in patients with antibiotic use or in patients with any diseases related to the partial or total depletion of the gut microbiota, and 2—stimulate gut microbiota growth will help in management of illness.

The compositions may be presented in unit-dose or multi-dose containers. And they may include additional ingredients that are conventional in the art with orally formulations. The composition of the invention may be in different galenical forms such as liquid or semiliquid, or capsule.

The invention will be illustrated below with the following examples, which are in no way restrictive.

TABLE 12
Probiotic enhancing composition. It includes mixed honey
or mixed honey extract alone or all the ingredients mixed
together, or mixed honey is mixed with any one of the ingredients
or mixed with two or more than two of the ingredients. One
or more pharmaceutically acceptable diluents or carriers
can be added to adjust the dose. Daily dose of the formulae
0.5-3 gram/kg.b.wt. Multiple composition can be prepared
by mixing two or more than two of the ingredients not including
mixed honey and propolis.

| | Doses per 70 kg.b.wt/day | | |
|---|---|---|---|
| | | | Example of |
| | Gram/ | | the formulae |
| Ingredients | kg.b.wt/day | Gram/day | gram |
| Mixed honey | 0.1-2 | 21-140 | 50 |
| or Extract with water, | 0.05-1 | | |
| alcohol or ethyl acetate | | | |
| Royal jelly or mixed | 0.014-0.85 | 1-6 | 5 |
| royal jelly | | | |
| Nigella sativa | 0.014-0.28 | 1-20 | 10 |
| Or Thymoquinone | 0.001-0.4 | 0.07-2.8 | 1 |
| Or Nigella sativa oil | 0.014-0.14 | 0.1-10 | 5 |
| Acacia Arabica | 0.07-0.42 | 1-30 | 10 |
| Garlic | 0.014-0.285 | 1-20 | 5 |
| Or aged garlic extract | 0.014-0.85 | 1-6 | 5 |
| Or garlic oil | 0.0014-0.071 | 0.1-5 | 0.5 |
| Propolis or mixed propolis | 0.011-0.285 | 0.8-20 | 5 |
| Or propolis extract | 0.005-0.21 | 0.4-15 | 5 |
| Or CAPE | 0.001-003 | 0.07-0.21 | 0.1 |
| Bee pollen or mixed bee pollen | 0.142-0.57 | 10-40 | 15 |
| Olive oil | 0.142-0.57 | 100-40 | 20 |
| Curcumin or curcumin extract | 0.014-0.42 | 1-30 | 20 |

Various modifications of the composition without departing from the scope may include incorporating new components to optimize efficacy or commercial processing, altering the percent weight of each constituent, introducing isolated chemical compounds derived from each ingredient individually or in combination to produce similar or better therapeutic results, or changing the formulation by subtracting aforementioned ingredients or using various combinations.

Some of the individual ingredients presented in the compositions were tested in growth of gut bacteria but the compositions that included mixture of the ingredients such as illustrated in the patent are not published or tested to stimulate growth of gut bacteria. Mixed honey, mixed propolis, mixed royal jelly and mixed bee pollen were never tested to stimulate growth of beneficial microorganisms.

### Vitiligo

Vitiligo is one of the most common cutaneous disorders of depigmentation, and no definitive cure currently exists. Traditional vitiligo treatments include topical and oral immunomodulators and phototherapy. Systemic corticosteroids are used in rapidly progressive cases to help with disease stabilization. The oral antibiotic minocycline has shown promise in the treatment of vitiligo due to its anti-inflammatory free-radical scavenging properties that confer a protective effect on melanocytes against H₂O₂—induced apoptosis. Statins and methotrexate might be useful. Pigmentation may result from melanocyte proliferation, melanogenesis, migration or increases in dendricity. Therefore, xepigmention of vitiligo requires melanocyte proliferation and migration. Keratinocytes have been shown to play a role in this process. The vitiligo healing composition offers the advantage of using ingredients of a natural origin which are readily available and whose association shows melanocyte-stimulating activity, excellent high nutritional value, wound healing properties, antioxidant and anti-inflammatory action, and antimicrobial and antiviral activity. It can be used alone with oral healing formulae or can be used with conventional moderate interventions.

The composition of the invention also offers the advantage of being easy to apply to the skin and/or mucous membranes or of being ingested by a living being. The invention is intended for the treatment of vitiligo of any cause or any type. The composition applied to the affected area and followed by sun exposure or ultraviolet exposure. The compositions may be presented in unit-dose or multi-dose containers. And they may include additional ingredients that are conventional in the art with topical formulations. The composition of the invention may be in different galenical forms such as in ointment, cream, or lotion form.

The invention will be illustrated below with the following examples, which are in no way restrictive.

TABLE 20
Vitiligo healing composition. It includes mixed honey or
mixed honey extract alone or all the ingredients mixed together,
or Nigella sativa is mixed with either bee venom or honey
alone, or bee venom is mixed with honey alone. One or more
pharmaceutically acceptable diluents or carriers can be
added to adjust the dose such as beeswax.

| | Concentrations | | |
|---|---|---|---|
| | | Ethanol or | Example of |
| Ingredients | Crude or Powder | water extract | formulae |
| Nigella sativa | 0.1-30% | 0.1-30% | 30% |
| or Thymoquinone | 0.1-10% | | Or 10% |
| | | | thymoquinone |
| Bee venom | 0.1-30 mg/100 g | mg/100 g | 30 mg/100 g |
| Mixed honey or mixed | 30-99% | | 50% |
| honey extract, such as | | | |
| water, ethanol or ethyl | | | |
| acetate | | | |

Various modifications of the composition without departing from the scope may include incorporating new components to optimize efficacy or commercial processing, altering the percent weight of each constituent, introducing isolated chemical compounds derived from each ingredient individually or in combination to produce similar or better therapeutic results, or changing the formulation by subtracting aforementioned ingredients or using various combinations. Some of the individual ingredient presented in the compositions was tested in melanocytes but the compositions that included mixture of the ingredients such as illustrated in the patent are not published or tested in vitiligo.

### Parasites

Oral or parenteral compositions are used as part of management of parasitic diseases. The oral composition includes:
Mixed honey: 0.3-2 g/kg.b.wt (or extract of honey at 0.15-1 g/kg).
Propolis or mixed propolis or their extracts: 0.02-0.14/kg.b.wt
Curcumin or curcumin extract: 0.014-0.40/kg.b.wt
Nigella sativa or its extract: 0.05-0.4/kg.b.wt
Garlic or its extract: 0.014-0.285/kg.b.wt
The extract of ingredients might include water extract, ethanol extract or ethyl acetate extracts or any other suitable extract.

This composition is intended to be part of management of parasitic diseases such as, not limited to, Entamoeba histolytica, Giardia lamella, Blastocystis species, schistosomiasis, malaria, flukes, trypanosome cruzi, hydatid cyst, toxoplasmosis, visceral leishmaniasis, ascaris, nematodes, trematodes, and trichomonas species. Mixed honey can be used alone or mixed with any of the above ingredients.

The parenteral composition includes:
Mixed honey: 0.1-2 g/k.b.wt (Honey extract can be used at 0.15-1 g/kg)
Propolis or mixed propolis extract: 0.1-30 mg/kg.b.wt
CAPE:0.001-10 mg/kg.b.wt or 0.1-500 micromole/kg.b.wt
Thymoquinone: 0.001-5 mg/kg.b.wt
Allicin: 1-5 mg/kg.b.wt
Curcumin or curcumin extract: 0.1-40mg/kg
The extract of ingredients might include water extract, ethanol extract or ethyl acetate extracts or any other suitable extract.

The parenteral composition is intended to be part of management of, not limited to, malaria, fluke, liver amoebiasis, trypanosome cruzi, hydatid cyst, toxoplasmosis and visceral or cutaneous leishmaniasis.

Accordingly, while only a few embodiments of the present invention have been shown and described, it is obvious that many changes and modifications may be made thereunto without departing from the spirit and scope of the invention.

What is claimed is:

1. A composition for treatment of a medical condition, comprising a mixture of at least two different types of honey or a mixture of extracts of at least two different types of honey, the two types of honey differing from each other in at least one of the following characteristics: color, floral origin, bee species, geographic region and season of harvest.

2. The composition according to claim 1, further comprising at least one of the following additional ingredients: propolis, mixed propolis, CAPE, bee venom, melittin, royal jelly, mixed royal jelly, bee pollen, mixed bee pollen, acacia Arabica, curcumin, nigella sativa, thymoquinone, clove, clove oil, olive oil, garlic, flaxseed, flaxseed oil, fish oil, green tea, fenugreek seeds, walnuts, date seeds, whey protein, peanuts, other nuts, and ginger.

3. The composition according to claim 1, wherein the honey mixture contains one honey in an amount of 45-55 wt % and a second honey in an amount of 45-55 wt %.

4. The composition according to claim 1, wherein the composition is formulated for topical application to the skin.

5. The composition according to claim 1, wherein the composition is configured for parenteral administration.

6. The composition according to claim 1, wherein the composition is configured for inhalation.

7. The composition according to claim 1, wherein the composition is configured for oral administration.

8. The composition according to claim 4, wherein the composition is in the form of a gel, ointment, spray, suppository, liquid or cream.

9. The composition according to claim 4, wherein the composition is disposed on a surface of a dressing for wound care.

10. The composition according to claim 1, wherein the mixed honey or extract of mixed honey is combined with propolis, mixed propolis or extracts of propolis or mixed propolis, and wherein the mixed honey or extract of mixed honey is present in an amount of between 50-99 wt % and the propolis or mixed propolis or extract of propolis or mixed propolis is present in an amount of 1-50% wt %.

11. The composition according to claim 2, wherein the composition is a wound healing composition configured for topical application and contains mixed honey in an amount of 10-99 wt % or extract of mixed honey in water, ethanol or ethyl acetate in an amount of 5-45%, and at least one of the following ingredients in proportions by weight:
Propolis (single or mixed): 1-30% or
CAPE: 1-1000 micromole/100 g or
extracts of propolis 1-15%;
Nigella sativa or extracts of nigella sativa: 0.1-15%
Thymoquinone: 0.1-5%;
Curcumin: 5-20% or curcumin extracts: 5-10%;
Garlic crude: 5-20% or as an extract: 1-10%;
Olive oil: 5-10%;
Bee pollen powder (single or mixed): 4-10%
or extract of bee pollen: 3-6%;
Cloves: 1-15% or extract of cloves: 0.5-10%;
Bee venom or extract: 0.1-15 mg/100 g or
Melittin or extract: 0.5-5 mg/100 g;
Acacia Arabica: 1-20%; and
Royal jelly (single or mixed): 1-20% or extract: 5-10%.

12. The composition according to claim 2, wherein the composition is configured for oral administration for treatment of kidney disease and includes the following ingredients in the following amounts given in grams per kilogram of body weight:
Mixed honey 0.1-2 or extract of mixed honey with water, alcohol or ethyl acetate 0.5-1;
Royal jelly or mixed royal jelly 0.014-0.085;
Nigella sativa 0.014-0.28, or Thymoquinone 0.001-0.4, or Nigella sativa oil 0.014-0.14;
Acacia Arabica 0.07-0.42;
Garlic 0.014-0.285; or aged garlic extract 0.014-0.14, or garlic oil 0.0014-0.071;
Propolis or mixed propolis 0.011-0.141 or propolis extract 0.005-0.141; Or CAPE 0.001-0.003;
Bee pollen or mixed bee pollen 0.142-0.57;
Olive oil 0.142-0.57;
Curcumin or curcumin extract 0.014-0.42;
Flaxseed 0.142-0.57 or flaxseed oil 0.014-0.085;
Ginger powder 0.014-0.285 or ginger water extract 0.014-0.285 or ginger ethanol extract 0.014-0.15;
Green tea or green tea extract 0.0071-0.071.

13. The composition according to claim 2, wherein the composition is configured for intravenous administration and contains the following ingredients in the following amounts:
Mixed honey or an extract of mixed honey in water, ethanol or ethyl acetate: 0.1-2 g/k.b.wt;
Propolis or mixed propolis or extract of propolis or mixed propolis: 0.1-30 mg/kg.b.wt;
CAPE: 0.001-10 mg/kg.b.wt or 0.1-500 micromole/kg.b.wt;
Thymoquinone: 0.001-5 mg/kg.b.wt; and
Curcumin or curcumin extract: 0.1-40 mg/kg.

14. A method for treating a medical condition comprising administering to a subject in need thereof a composition comprising a mixture of at least two different types of honey, the two types of honey differing from each other in at least one of the following characteristics: color, floral origin, bee species, geographic region and season of harvest.

15. The method according to claim 14, wherein the composition is administered orally.

16. The method according to claim 14, wherein the composition is administered by inhalation.

17. The method according to claim 14, wherein the composition is administered parenterally.

18. The method according to claim 14, wherein the composition is administered topically.

19. The method according to claim 14, wherein the composition additionally contains at least one of the following ingredients: propolis, mixed propolis, CAPE, bee venom, melittin, royal jelly or mixed royal jelly, bee pollen or mixed bee pollen, acacia Arabica, curcumin, nigella sativa, thymoquinone, cloves or clove oil, olive oil, fish oil, garlic, flaxseed or flaxseed oil, green tea, fenugreek seeds, walnuts, date seed, whey protein, peanuts, other nuts, and ginger.

20. The method according to claim 19, wherein the medical condition is one selected from the group consisting of wounds, burns, ulcers, skin infection, contact dermatitis, eczema, psoriasis, wrinkled'skin, allergic reactions, herpes simplex, herpes zoster, cutaneous leishmaniasis, c. diff colitis, peritonitis, proteinuria, urinary calculus, anemia, for potentiating the effects of antibiotics and chemotherapy, dyslipidemia, diabetes mellitus, cardiovascular diseases, H. pylori gastritis or ulcers, dyspepsia, acute and chronic kidney diseases, hypertension, amelioration of toxic effects of heavy metals or chemotherapies or radio therapy, infectious diseases, sepsis, dehydration, malnutrition, sinusitis, lung obstructive diseases and malignancies, and as a sweetener.
